(19) United States
(12) Patent Application Publication
(10) Pub. No.: US 2012/0029282 A1
(43) Pub. Date: Feb. 2, 2012

(54) GUIDE ASSEMBLY FOR ENDOSCOPE

(76) Inventors: Shinichi YAMAKAWA, Kanagawa (JP); Takayuki NAKAMURA, Kanagawa (JP); Masayuki IWASAKA, Kanagawa (JP)

(21) Appl. No.: 13/193,674

(22) Filed: Jul. 29, 2011

(30) Foreign Application Priority Data
Jul. 30, 2010 (JP) 2010-171919

(51) Int. Cl. A61B 1/00

(52) U.S. Cl. 600/114

(57) ABSTRACT

A guide assembly of a self-propelled type for an endoscope having an elongated tube for entry in a body cavity is provided. First and second self-propelled units are secured to a steering device provided in the elongated tube, arranged in an axial direction of the elongated tube, the second self-propelled unit receiving force for driving from an external drive source. A flexible connector connects the second self-propelled unit to the first self-propelled unit and transmits the force applied to the second self-propelled unit to the first self-propelled unit. Preferably, the connector includes at least two coil springs having diameters different from one another, and combined in such a multi layer form that winding directions thereof are opposite to one another. The force is torque in a rotational direction about an axis of the axial direction.

### BACKGROUND OF THE INVENTION

1. Field of the Invention

The present invention relates to a guide assembly for an endoscope. More particularly, the present invention relates to a guide assembly capable of causing an endoscope to enter a body cavity smoothly, and keeping a steering device in the endoscope steerable readily even with a self-propelled structure for guiding.

2. Description Related to the Prior Art

An endoscope is used to diagnose a body cavity, such as a large intestine in a gastrointestinal tract. Manipulation of the endoscope is a difficult process, because the large intestine is a tortuous organ in a human body, and some body parts are very changeable in the position in the body, such as a sigmoid colon and a transverse colon. Learning the manipulation of the endoscope of the large intestine requires much experience and time. If a doctor is insufficiently skilled in the manipulation, physical load to the body of a patient will be very large.

U.S. Pat. Nos. 6,971,990 and 7,736,300 (corresponding to JP-A 2009-513250) disclose a self-propelled apparatus for propelling the endoscope in the axial direction in the body cavity to facilitate the manipulation even for an unskilled operator or doctor. The self-propelled apparatus of the documents includes a movable endless track device or crawler device or toroidal device. The endless track device is driven to turn around for the endoscope to travel mechanically. Force of propulsion is created by the endless track device contacting a wall of the large intestine, so as to guide the endoscope deeply in the body cavity.

However, U.S. Pat. Nos. 6,971,990 and 7,736,300 disclose the self-propelled apparatus in which a support or housing of the endless track device longitudinally extends in the axial direction of the elongated tube. There is a problem in that the steering of the steering device is obstructed by the combined use of the guide assembly or the self-propelled apparatus, and that flexibility of the elongated tube may be lower. Accordingly, the manipulation may be more difficult.

### SUMMARY OF THE INVENTION

In view of the foregoing problems, an object of the present invention is to provide a guide assembly capable of causing an endoscope to enter a body cavity smoothly, and keeping a steering device in the endoscope steerable readily even with a self-propelled structure for guiding.

In order to achieve the above and other objects and advantages of this invention, a guide assembly of a self-propelled type for an endoscope having a section of an elongated tube for entry in a body cavity is provided, the elongated tube having a steering device on a distal side with a variable direction. At least first and second self-propelled units are mounted about the steering device, arranged with one another, for propulsion in an axial direction of the elongated tube by contacting a wall of the body cavity, the second self-propelled unit receiving force for driving from an external drive source. A flexible connector connects the second self-propelled unit to the first self-propelled unit, and transmitting the force applied to the second self-propelled unit to the first self-propelled unit.

The connector includes at least two coils having diameters different from one another, and combined by containing a first one in a second one thereof so that winding directions thereof are opposite to one another.

The force is torque in a rotational direction about an axis of the axial direction. Each of the at least first and second self-propelled units includes a first sleeve, secured around the steering device, and rotated thereabout by the torque. A driving device is connected with the first sleeve, for converting the torque into force in the axial direction.

Each of the at least first and second self-propelled units includes an endless track device, having an annular surface, driven by the driving device when the torque is applied to the first sleeve, for turning around on an endless track in the axial direction.

The first sleeve is constituted by a worm gear. Furthermore, a bearing sleeve is mounted around the steering device, for supporting the worm gear in a rotatable manner about the axis. The driving device includes an engagement roller, having teeth, rotatable about an axis crosswise to the axial direction, meshed with the worm gear, for moving the endless track device.

The driving device further comprises a first ring sleeve disposed about the worm gear. A first through opening is formed through a wall of the first ring sleeve, for supporting the engagement roller in a rotatable manner. A second ring sleeve is disposed about the first ring sleeve, for supporting the endless track device movably. A second through opening is formed through a wall of the second ring sleeve. An idler roller is secured in the second through opening, for rotating about an axis crosswise to the axial direction, and nipping the endless track device in cooperation with the engagement roller.

The idler roller is constituted by a pair of idler rollers, and the engagement roller is disposed between the idler rollers.

Furthermore, a flexible tubular cover covers the at least two coils.

The endless track device is formed from fluid-impermeable material, and internally charged with liquid.

In a preferred embodiment, the endless track device is formed from fluid-impermeable material, and internally charged with gel.

The endless track device is formed from biocompatible plastic material.

Preferably, the second self-propelled unit is controlled remotely.

Preferably, the at least two coils are three coils.

Preferably, the connector is single.

Consequently, it is possible to keep a steering device in the endoscope steerable readily even with a self-propelled structure for guiding, because the flexible connector connects the second self-propelled unit to the first self-propelled unit suitably.

### BRIEF DESCRIPTION OF THE DRAWINGS

The above objects and advantages of the present invention will become more apparent from the following detailed description when read in connection with the accompanying drawings, in which:

FIG. 1 is a plan illustrating an endoscope system;

FIG. 2 is a perspective view illustrating a guide assembly;

FIG. 3 is an exploded perspective view illustrating the guide assembly;

FIG. 4 is a vertical section illustrating the guide assembly.

### DETAILED DESCRIPTION OF THE PREFERRED EMBODIMENT(S) OF THE PRESENT INVENTION

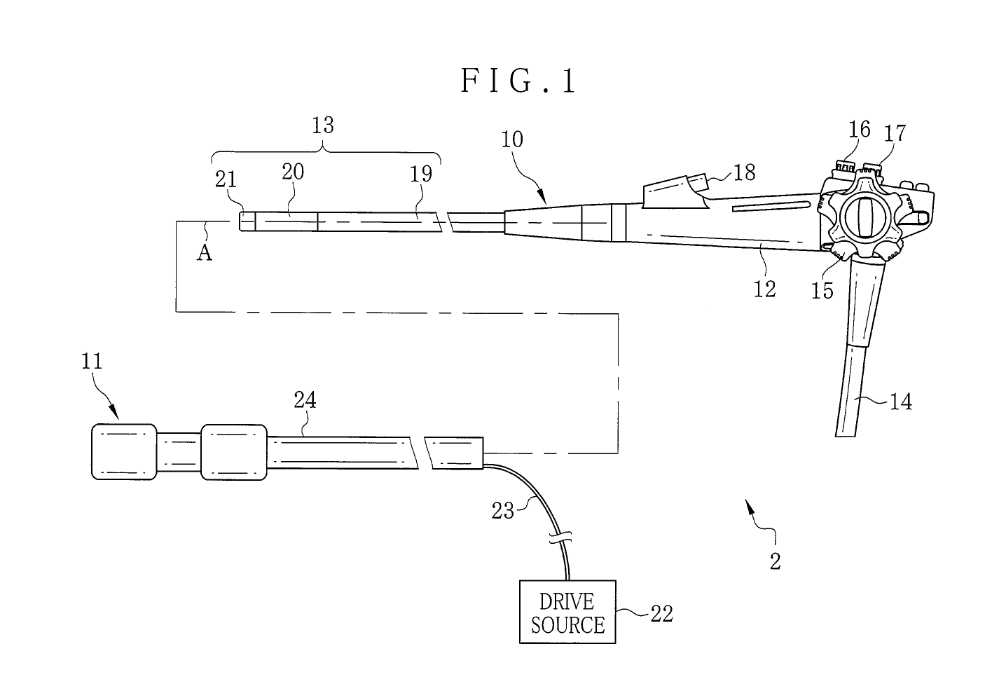

In FIG. 1, an endoscope system 2 includes an endoscope 10 and a self-propelled type of guide assembly 11. The endoscope 10 has a handle device 12 and an elongated tube 13 or guide tube disposed to extend from the handle device 12 for entry in a body cavity, for example, a large intestine of a gastrointestinal tract. A universal cable 14 is disposed to extend from the handle device 12. Connection plugs (not shown) are disposed at a proximal end of the universal cable 14 for connection with a light source apparatus and a processing apparatus (not shown) in a removal manner.

The handle device 12 includes steering wheels 15, an air/water button 16 and a suction button 17. The air/water button 16 is operable for supplying air or water through a distal end of the elongated tube 13. An instrument channel 18 is formed through the handle device 12 and the elongated tube 13 for receiving entry of a forceps, electrocautery device or other medical instrument.

The elongated tube 13 includes a flexible device 19, a steering device 20 and a head assembly 21 in a sequence in a distal direction from the handle device 12. The flexible device 19 has a length as great as several meters for reach of the head assembly 21 to an object of interest in a body cavity. The steering device 20 bends up and down and to the right and left in response to operation of the steering wheels 15 of the handle device 12. Thus, the head assembly 21 can be steered in a desired direction in the patient's body.

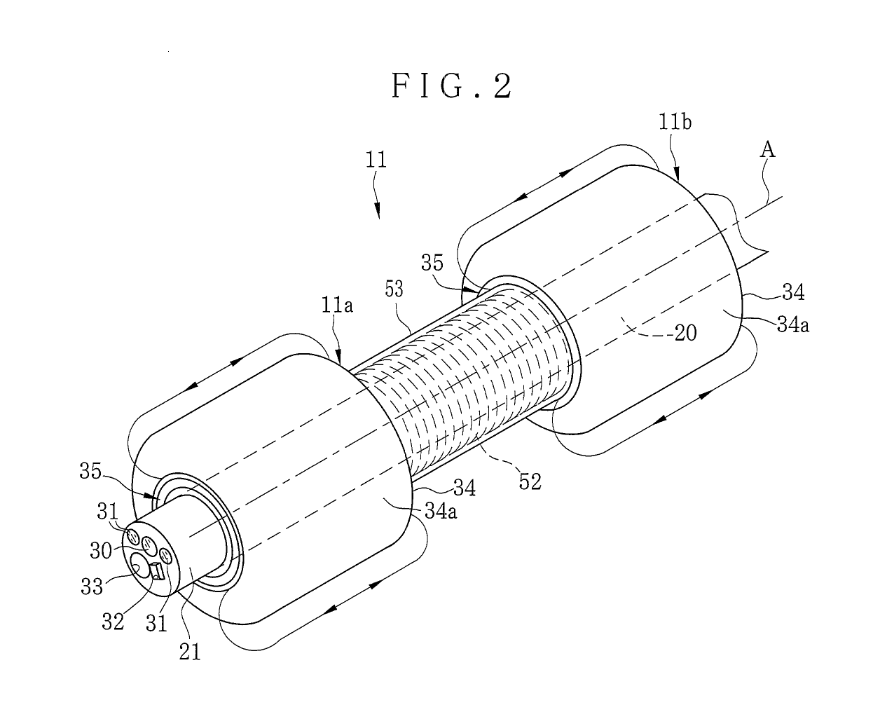

An imaging window 30 is formed in the head assembly 21 for imaging of a body part in the body. See FIG. 2. The head assembly 21 contains objective optics and an image sensor or solid-state image pickup device for imaging, such as CCD and CMOS image sensors. The image pickup device is connected to the processing apparatus by a signal line, which extends through the elongated tube 13, the handle device 12 and the universal cable 14. An object image of the body part is focused on a reception surface of the image pickup device, and is converted into an image signal. The processing apparatus processes the image signal from the image pickup device through the signal line by image processing, and obtains a video signal by conversion after the image processing. The object image is output and displayed on a monitor display panel (not shown) according to the video signal.

Various openings are formed in the head assembly 21 as illustrated in FIG. 2. Among those, a lighting window 31 passes illumination light from a light source apparatus toward an object of interest. An air/water nozzle 32 supplies air or water toward the imaging window from an air/water supply device in the light source apparatus in response to depression of the air/water button 16. An instrument opening 33 causes a distal end of a medical instrument from the instrument channel 18 to appear distally.

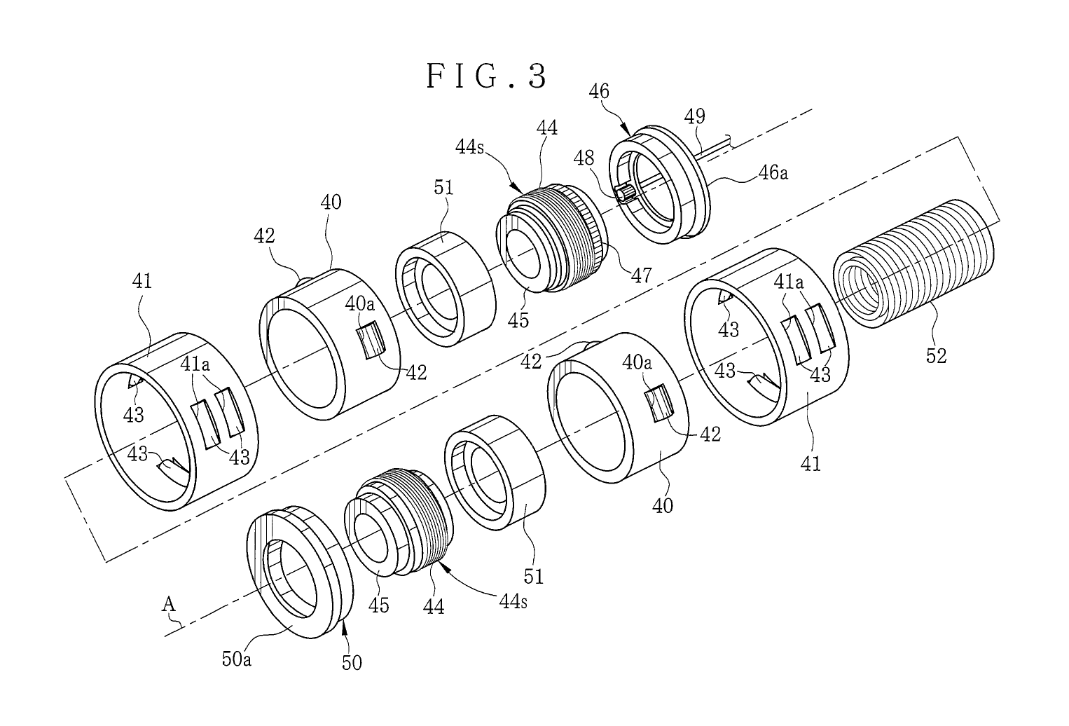

The guide assembly 11 is a device mounted on the endoscope 10, for assisting forward and backward movement of the elongated tube 13 of the endoscope 10 in a body cavity. A drive source 22 or motor drives the guide assembly 11. A torque coil structure 49 of a multi component type or three component type is connected with the drive source 22, and transmits torque to the guide assembly 11. See FIG. 3. There is a protection sheath 23 through which the torque coil structure 49 is entered at its full length. The torque coil structure 49 rotates in the protection sheath 23 when the drive source 22 is actuated.

An overtube 24 is used to cover the elongated tube 13, and is ready to expand and shrink in an axial direction of an axis A of the elongated tube 13. The protection sheath 23 of the torque coil structure 49 is entered between the overtube 24 and the elongated tube 13.

A controller (not shown) controls the drive source 22. A button panel (not shown) is connected to the controller. The button panel includes a command button for inputting command signals for forward movement, backward movement and stop of the self-propelled type of guide assembly 11, and a speed button for changing a moving speed of the guide assembly 11. Note that a control program can be prepared suitably for an object to be imaged. The drive source 22 can be actuated according to the control program without manipulating the button panel, so as to actuate the guide assembly 11 automatically.

In FIG. 2, the guide assembly 11 includes a first self-propelled unit 11a and a second self-propelled unit 11b or guide structures. The first self-propelled unit 11a is positioned on a distal side from the second self-propelled unit 11b. Those are secured to the steering device 20 of the elongated tube 13 of the endoscope 10. Each of the self-propelled units 11a and 11b includes a movable endless track device 34 or crawler device or toroidal device, and a driving device 35 or support device or barrel device with a rotating mechanism. The endless track device 34 has a hollow shape with an annular surface, is movable on an endless track, and is formed from a biocompatible plastic material having flexibility and fluid-impermeability. An example of the biocompatible plastic material is polyvinyl chloride. Also, polyamide resin, fluorocarbon resin, polyurethane resin and the like can be used. The inside of the endless track device 34 is filled with suitable fluid, such as liquid, gel, gas, or a combination of at least two of liquid, gel and gas.

The endless track device 34 of each of the self-propelled units 11a and 11b endlessly turns around in the axial direction of the axis A. The endless track device 34 of the first self-propelled unit 11a is turned in synchronism with turn around of the endless track device 34 of the second self-propelled unit 11b. When an outer surface 34a of the endless track device 34 contacts an inner wall of a body cavity, propelling force occurs in the self-propelled units 11a and 11b to move the elongated tube 13 along the axis A.

To propel the elongated tube 13 in the distal direction, the elongated tube 13 is moved by the turn around of the endless track device 34 in an endless manner to orient the outer surface 34a in the proximal direction. To move the elongated tube 13 in the proximal direction, the endless track device 34 is turned around endlessly to orient the outer surface 34a in the distal direction.

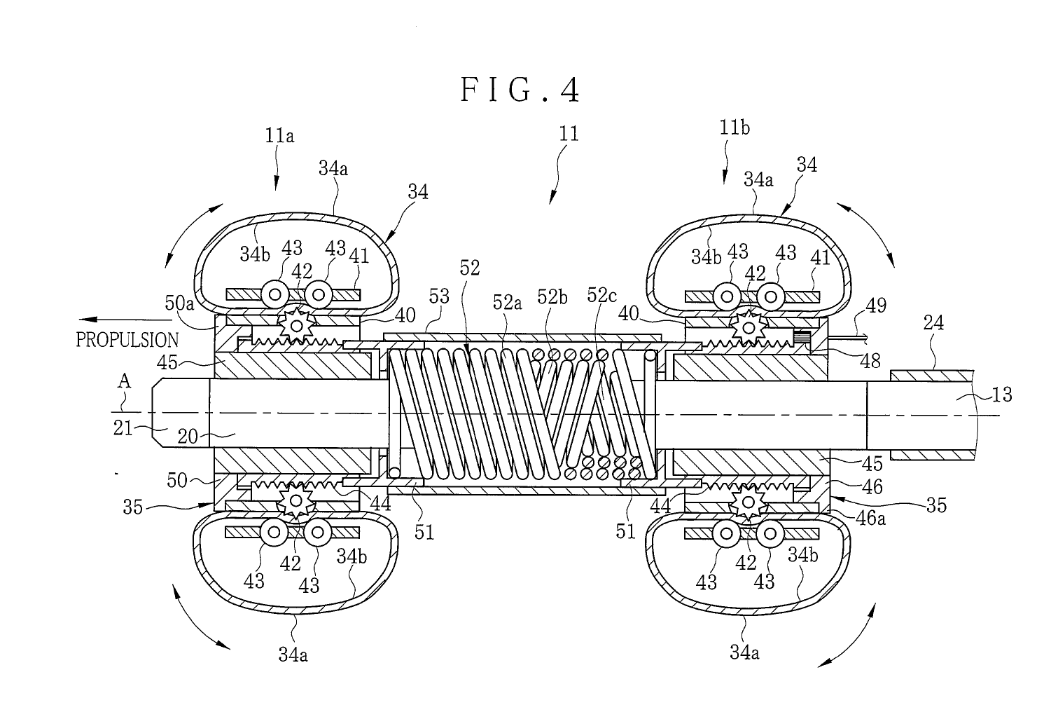

In FIGS. 3 and 4, the driving device 35 includes a first ring sleeve 40 and a second ring sleeve 41. Both of the first and second ring sleeves 40 and 41 are cylindrical, and have an equal size along the axis A. A diameter of the first ring sleeve 40 is smaller than that of the second ring sleeve 41. The first ring sleeve 40 is contained in and surrounded by the second ring sleeve 41. In FIG. 3, the endless track device 34 is not depicted.

Through openings 40a are formed in a wall of the first ring sleeve 40. An engagement roller 42 or drive roller or toothed roller has teeth, is disposed inside each of the through openings 40a, and rotates about an axis perpendicular to the axis A. The engagement roller 42 is disposed at the middle of the first ring sleeve 40 in the direction of the axis A. Three engagement rollers 42 are arranged at a pitch of 120 degrees in the circumferential direction of the first ring sleeve 40.

Through openings 41a are formed in a wall of the second ring sleeve 41. A pair of idler rollers 43 or driven rollers are disposed inside respectively the through openings 41a. Each of the idler rollers 43 is rotatable about an axis parallel to the axis of the engagement roller 42. Three pairs of idler rollers 43 are arranged at a pitch of 120 degrees in the circumferential direction of the second ring sleeve 41. When the first ring sleeve 40 is contained in the second ring sleeve 41, the second ring sleeve 41 is positioned relative to the first ring sleeve 40 to set the engagement roller 42 between the idler rollers 43. The endless track device 34 is mounted about the second ring sleeve 41 by passage in its end openings. The endless track device 34 is squeezed between the engagement roller 42 and the idler rollers 43. An inner surface 34b of the endless track device 34 is contacted by the idler rollers 43, which are rotated by endless turn around of the endless track device 34.

Specifically, the endless track device 34 is prepared in the following manner. At first, a plastic tube having two open ends with flexibility and elasticity is initially formed from a sheet or film of the above-described suitable material. The plastic tube is halfway inserted in a sleeve lumen of the second ring sleeve 41. Then a portion of the plastic tube outside the sleeve lumen is bent back externally and extended to cover the periphery of the second ring sleeve 41. A first side line of the inserted half of the plastic tube is opposed to a second side line of the bent half to the plastic tube, so that the halves are attached together along the first and second side lines by adhesion, welding or other suitable method. Finally, the toroidal shape of the endless track device 34 is obtained.

A worm gear 44 or worm thread is contained in the first ring sleeve 40. There is a bearing sleeve 45 or holding sleeve for supporting a worm gear sleeve 44s (worm drive or worm sleeve as a first sleeve) including the worm gear 44. The elongated tube 13 of the endoscope 10 is entered in the bearing sleeve 45 which becomes mounted fixedly on the elongated tube 13. As the bearing sleeve 45 is positioned on the elongated tube 13, the head assembly 21 projects distally from the bearing sleeve 45 of the first self-propelled unit 11a. A worm thread of the worm gear 44 rotates about the bearing sleeve 45 in bearing contact along the axis A. The worm gear 44 is meshed with the engagement roller 42, which is rotated by the worm gear 44.

A rear end ring 46 is attached to the first ring sleeve 40 of the second self-propelled unit 11b. A flange 46a is a portion of the rear end ring 46 at its peripheral edge. The flange 46a, when the rear end ring 46 is attached to the first ring sleeve 40, contacts a rear edge of the first ring sleeve 40. An end of the bearing sleeve 45 is fitted in an inner hole of the rear end ring 46 in a tight manner without dropping.

Spur gear teeth 47 or a driven gear is formed with a proximal end of the worm gear 44 inside the first ring sleeve 40 of the second self-propelled unit 11b, the teeth being arranged about the axis A. A pinion 48 is secured to the rear end ring 46 in a rotatable manner. An axis of the pinion 48 is parallel to the axis A. The pinion 48 is meshed with the spur gear teeth 47, and is firmly connected with the torque coil structure 49. Thus, the pinion 48 is rotated by rotation of the torque coil structure 49. The torque coil structure 49 includes three coil springs combined in a multi layer form in such a state that their winding directions are different from one another. The torque coil structure 49 can transmit torque even upon rotating in any of the forward and backward directions. When the pinion 48 rotates, the spur gear teeth 47 rotate responsively.

A front end ring 50 is attached to the first ring sleeve 40 of the first self-propelled unit 11a. A flange 50a is a portion of the front end ring 50 at its peripheral edge. The flange 50a, when the front end ring 50 is attached to the first ring sleeve 40, contacts a front edge of the first ring sleeve 40. An end of the bearing sleeve 45 is fitted in an inner hole of the front end ring 50 in a tight manner without dropping.

A proximal end of the worm gear 44 in the first self-propelled unit 11a has an outer annular recess. A distal end of the worm gear 44 in the second self-propelled unit 11b has an outer annular recess. A connecting ring 51 is fitted in each outer annular recess of the worm gear 44 on a suitable side. There is a connecting coil structure 52 of a multi component type or three component type. Each of ends of the connecting coil structure 52 is fitted on the connecting ring 51. The connecting ring 51 and the connecting coil structure 52 rotate together with the worm gear 44. Thus, torque of the worm gear 44 in the second self-propelled unit 11b is transmitted to the worm gear 44 of the first self-propelled unit 11a.

The connecting coil structure 52 includes a first coil spring 52a, a second coil spring 52b and a third coil spring 52c. The first coil spring 52a is positioned externally. The second coil spring 52b has an outer diameter substantially equal to an inner diameter of the first coil spring 52a. The third coil spring 52c has an outer diameter substantially equal to an inner diameter of the second coil spring 52b. The coil springs 52a, 52b and 52c are combined in a multi layer form in such a state that their winding directions are different from one another. Specifically, the first and third coil springs 52a and 52c have turns wound in the counterclockwise direction. The second coil spring 52b has turns wound in the clockwise direction.

When the connecting coil structure 52 is rotated in the counterclockwise direction by rotation of the connecting ring 51, the first and third coil springs 52a and 52c are shifted and tightened in an inward direction, the second coil spring 52b being shifted and loosened in an outward direction. Thus, torque can be transmitted with high efficiency owing to the tight contact between the first and second coil springs 52a and 52b. When the connecting coil structure 52 is rotated in the clockwise direction by rotation of the connecting ring 51, the first and third coil springs 52a and 52c are shifted and loosened in the outward direction, the second coil spring 52b being shifted and tightened in the inward direction. Thus, torque can be transmitted with high efficiency owing to the tight contact between the second and third coil springs 52b and 52c. Note that a structure and operation of the torque coil structure 49 are basically the same as the connecting coil structure 52.

A tubular cover 53 is flexible along the axis A of the elongated tube 13, and has one end to which the connecting ring 51 is secured. The tubular cover 53 covers the connecting coil structure 52, and prevents body fluid from contacting the connecting coil structure 52. In FIG. 3, the tubular cover 53 is not depicted.

The operation of the endoscope system 2 is described now. At first, the overtube 24 is retained on the elongated tube 13 of the endoscope 10. The elongated tube 13 is entered in the bearing sleeve 45 to mount the guide assembly 11 on the elongated tube 13.

After securing the overtube 24 and the guide assembly 11 to the endoscope 10, a power source of the processing apparatus, light source apparatus and controller is turned on. Then personal information of the patient is input. The elongated tube 13 of the endoscope 10 is entered in his of her body cavity.

After the head assembly 21 is advanced to a predetermined body part, for example, slightly short of a sigmoid colon, then the button panel is operated to turn on a power source for the drive source 22 of the self-propelled type of guide assembly 11. Then a command signal for start is input with the button panel. The drive source 22 rotates the torque coil structure 49 in a predetermined direction. In the second self-propelled unit 11b, the pinion 48 is rotated by rotation of the torque coil structure 49. The worm gear 44 is rotated by the pinion 48. Thus, the connecting coil structure 52 rotates together with the worm gear 44 in the second self-propelled unit 11b, to shift the worm gear 44 of the first self-propelled unit 11a inwards for firm contact. Accordingly, transmission of torque to the worm gear 44 of the first self-propelled unit 11a can be efficient, because the torque of the worm gear 44 of the second self-propelled unit 11b is transmitted by the connecting coil structure 52.

When the worm gear 44 rotates together with the self-propelled units 11a and 11b, the engagement roller 42 is rotated responsively. Thus, the endless track device 34 endlessly turns around in each of the self-propelled units 11a and 11b. The guide assembly 11 advances in the axis direction of the elongated tube 13 when the outer surface 34a of the endless track device 34 contacts a wall of a body cavity. Consequently, the head assembly 21 advances along the wall of the body cavity.

When a command signal for a change is input by operating the button panel, the drive source 22 changes a rotational speed of the torque coil structure 49. Thus, a moving speed of the self-propelled type of guide assembly 11 is changed. When a command signal for return is input by operating the button panel, the drive source 22 causes the torque coil structure 49 to rotate in a backward direction, to move the guide assembly 11 and the head assembly 21 backwards. When a command signal for a stop is input by operating the button panel, the drive source 22 stops to stop moving the guide assembly 11. It is possible to propel the head assembly 21 through the body cavity to an object of interest by suitably repeating those steps of the movement.

A doctor or operator steers the steering device 20 of the endoscope 10 by manipulating the steering wheels 15, to bend the head assembly 21 in a desired direction. As the second self-propelled unit 11b is connected to the first self-propelled unit 11a by the connecting coil structure 52 in the guide assembly 11, the connecting coil structure 52 bends together with the steering device 20 according to the flexibility, for the self-propelled units 11a and 11b to follow the steering of the steering device 20. This is effective in keeping the steering device 20 free from being obstructed by the guide assembly 11.

In the embodiment, the connecting coil structure 52 is used between the two worm gears 44 in the self-propelled units 11a and 11b. However, other elements may be used for transmitting torque of the second self-propelled unit 11b to the first self-propelled unit 11a and having flexibility along the axis A, for example, only one coil spring, a rubber tube or the like.

Although the self-propelled units 11a and 11b are disclosed the above embodiment, the number of the self-propelled units or guide structures may be three or more. Also, the number of the drive sources or motors for the self-propelled units may be two or more.

In the above embodiments, the self-propelled type of guide assembly is used with the endoscope for a medical use. Also, the guide assembly of the invention can be used with an endoscope for industrial use, an ultrasonic probe, or other instruments for imaging in a cavity. Although the movable endless track device or crawler device or toroidal device is turned around in the guide assembly, a guide assembly of the invention can be any mechanical type for entry in a body cavity as a component for an instrument for imaging.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A guide assembly of a self-propelled type for an endoscope having a section of an elongated tube for entry in a body cavity, said elongated tube having a steering device on a distal side with a variable direction, comprising:
at least first and second self-propelled units, mounted about said steering device, arranged with one another, for propulsion in an axial direction of said elongated tube by contacting a wall of said body cavity, said second self-propelled unit receiving force for driving from an external drive source; and
a flexible connector for connecting said second self-propelled unit to said first self-propelled unit, and transmitting said force applied to said second self-propelled unit to said first self-propelled unit.

2. A guide assembly as defined in claim 1, wherein said connector includes at least two coils having diameters different from one another, and combined by containing a first one in a second one thereof so that winding directions thereof are opposite to one another.

3. A guide assembly as defined in claim 2, wherein said force is torque in a rotational direction about an axis extending in said axial direction;
each of said at least first and second self-propelled units includes:
a first sleeve, secured around said steering device, and rotated thereabout by said torque;
a driving device, connected with said first sleeve, for converting said torque into force in said axial direction.

4. A guide assembly as defined in claim 3, wherein each of said at least first and second self-propelled units includes an endless track device, having an annular surface, driven by said driving device when said torque is applied to said first sleeve, for turning around in said axial direction.

5. A guide assembly as defined in claim 4, wherein said first sleeve is constituted by a worm gear sleeve having a worm gear;
further comprising a bearing sleeve, mounted around said steering device, for supporting said worm gear sleeve in a rotatable manner about said axis;
said driving device includes an engagement roller, having teeth, rotatable about an axis crosswise to said axial direction, meshed with said worm gear sleeve, for moving said endless track device.

6. A guide assembly as defined in claim 5, wherein said driving device further comprises:
a first ring sleeve disposed about said worm gear sleeve;
a first through opening, formed through a wall of said first ring sleeve, for supporting said engagement roller in a rotatable manner;
a second ring sleeve, disposed about said first ring sleeve, for supporting said endless track device movably;
a second through opening formed through a wall of said second ring sleeve;
a first roller, secured in said second through opening, for rotating about an axis crosswise to said axial direction, and nipping said endless track device in cooperation with said engagement roller.

7. A guide assembly as defined in claim 6, wherein said first roller is constituted by a pair of first rollers, and said engagement roller is disposed between said first rollers.

8. A guide assembly as defined in claim 4, further comprising a flexible tubular cover for covering said at least two coils.

9. A guide assembly as defined in claim 4, wherein said endless track device is formed from fluid-impermeable material, and internally charged with liquid.

10. A guide assembly as defined in claim 4, wherein said endless track device is formed from fluid-impermeable material, and internally charged with gel.

11. A guide assembly as defined in claim 4, wherein said endless track device is formed from biocompatible plastic material.

12. A guide assembly as defined in claim 3, wherein said second self-propelled unit is controlled remotely.

13. A guide assembly as defined in claim 3, wherein said at least two coils are three coils.
